(19) United States
(12) Patent Application Publication
(10) Pub. No.: US 2014/0224897 A1
(43) Pub. Date: Aug. 14, 2014

(54) APPLICATOR DEVICE FOR LIQUID OR VOLATILE PRODUCTS

(71) Applicants: Guillaume GARAGNON, Portes-Les-Valence (FR); Olivier BLANC, Saint-Peray (FR)

(72) Inventors: Guillaume GARAGNON, Portes-Les-Valence (FR); Olivier BLANC, Saint-Peray (FR)

(21) Appl. No.: 14/257,020

(22) Filed: Apr. 21, 2014

Related U.S. Application Data
(63) Continuation of application No. 14/004,452.

(30) Foreign Application Priority Data
Mar. 11, 2011 (FR) 1152015

(51) Int. Cl. A45D 34/04; A61L 9/12

(52) U.S. Cl. CPC A45D 34/04; A61L 9/12; A45D 2034/005
USPC 239/43; 401/196; 401/202

(57) ABSTRACT

The invention relates to an applicator device for liquid or volatile products, having a product reservoir (1), a neck (14) of which contains a part (16) for diffusing or applying the product; a cartridge (2), provided with a neck (24), for holding the reservoir (1), the cartridge being designed such that the reservoir can take up a first position in which the application and diffusion part does not protrude from the neck and a second position in which this part protrudes from the neck; and a receptacle (3) for the cartridge (2), said receptacle being provided with means for causing the reservoir to move from the first position to the second when the cartridge is in the receptacle.

### CROSS-REFERENCE TO RELATED APPLICATIONS

The present application is a continuation of U.S. National Stage Patent Application Serical Number 14/004,452, filed Sep. 11, 2013, which is a National Stage of PCT International Application Serial Number PCT/FR2012/050468, filed Mar. 7, 2012, which claims priority under 35 U.S.C. §119 of French Patent Application Serial Number 11/52015, filed Mar. 11, 2011, the disclosures of which are incorporated by reference herein.

### BACKGROUND

The present invention generally relates to devices for applying or diffusing liquid or volatile products and, more specifically, to a perfume application device.

### DISCUSSION OF THE RELATED ART

Perfume application devices can be distributed in two large categories. A first category is formed of sprayers. A second category to which the present invention applies is formed of applicators or diffusers based on the use of a part made of felt or the like, impregnated with perfume.

Such devices generally comprise a perfume reservoir having an opening in contact with a side of a felt part, the other side of the felt being accessible from the outside of the device. Such devices may be used either as room fragrance diffusers, the accessible portion of the felt then being in the open air, or as a perfume applicator, the user using the felt as an applicator pad.

A difficulty of the use of such felts is to stop the perfume diffusion or evaporation via the felt between periods of use. It is then necessary to provide a cover covering the accessible portion of the felt.

Rechargeable devices are further more and more often required. A difficulty then is to protect the felt part of the recharge as long as this recharge is not in the device. This difficulty is increased when a rechargeable device capable of receiving recharges containing different perfumes is desired to be provided. The felt should then be changed at the same time as the recharge.

Similar problems are posed in different fields where it is desired to use a part for diffusing or applying a volatile liquid product in a rechargeable device. Such is for example the case for certain inks, glues, or liquid foundations.

### SUMMARY OF THE INVENTION

An object of an embodiment of the present invention is to overcome all or part of the disadvantages of usual rechargeable devices for applying or diffusing liquids or volatile products.

Another object of an embodiment of the present invention is to provide a rechargeable device where the application part is protected (non-usable) as long as the recharge is not in the rechargeable device.

Another object of an embodiment of the present invention is to provide a device which is simple to manufacture.

To achieve all or part of these and other objects, the present invention provides an applicator device for volatile products comprising:

a product reservoir having a neck containing a part for applying or diffusing the product;

a cartridge for receiving the reservoir, provided with a neck, the cartridge being adapted for the reservoir to take a first position where the application or diffusion part does not protrude from the neck, and a second position where this part protrudes from the neck; and

a receptacle for the cartridge, provided with means for having, when the cartridge is in the receptacle, the reservoir move from the first to the second position.

According to an embodiment of the present invention, said means comprise at least one pin protruding from a shaft comprised in the receptacle, the pin being intended to cross an opening of the cartridge to drive the reservoir to the second position.

According to an embodiment of the present invention, the bottoms of the cartridge and of the shaft of the receptacle comprise elements capable of cooperating by magnetization when the reservoir is in the second position.

According to an embodiment of the present invention, said pin is threaded, said orifice of the cartridge being tapped.

According to an embodiment of the present invention, said means comprise a horizontally-mobile wedge capable of cooperating with a rod protruding from the bottom of the reservoir and crossing the bottom of the cartridge.

According to an embodiment of the present invention, the reservoir and the cartridge have a conical shape, the cartridge wall being more flexible than the wall of the reservoir so that a pressure on the cartridge wall causes an axial displacement of the reservoir.

According to an embodiment of the present invention, said means comprise a horizontally-mobile pin capable of crossing a vertical port of the cartridge to bear against the bottom of the reservoir.

According to an embodiment of the present invention, said means can be actuated by a control member accessible from the outside of the receptacle.

According to an embodiment of the present invention, the bottoms of the reservoir and of the cartridge comprise elements capable of cooperating by magnetization when the reservoir is in the first position.

According to an embodiment of the present invention, the bottom of the cartridge comprises at least one magnet, the bottom of the reservoir comprising at least one metal part opposite to this magnet.

According to an embodiment of the present invention, said magnet is housed in a through opening of the bottom of the cartridge and is capable of cooperating with a metal bottom of the receptacle shaft.

According to an embodiment of the present invention, the reservoir, the cartridge, and the shaft of the receptacle have an approximately cylindrical shape.

According to an embodiment of the present invention, the application or diffusion part is a felt

According to an embodiment of the present invention, the reservoir contains perfume.

According to an embodiment of the present invention, the receptacle comprises a removable cover.

According to an embodiment of the present invention, in the first position, the reservoir rests, directly or via spacers, on the bottom of the cartridge.

### BRIEF DESCRIPTION OF THE DRAWINGS

The foregoing and other objects, features, and advantages will be discussed in detail in the following non-limiting description of specific embodiments in connection with the accompanying drawings, among which:

FIGS. 1A, 1B, and 1C are simplified perspective views of components of an embodiment of a rechargeable perfume application or diffusion device;

FIG. 2 is a cross-section view of an embodiment corresponding to FIG. 1B;

FIG. 3 is a partial cross-section view of the device of FIG. 1C;

FIG. 3A is a perspective cross-section view of a cartridge of FIGS. 1B and 2 in a situation of use in a device;

FIGS. 4A and 4B very schematically illustrate an alternative embodiment of the means for having, when the cartridge is in the receptacle, the reservoir move from the first to the second position;

FIGS. 5A and 5B very schematically illustrate another alternative embodiment of the means for having, when the cartridge is in the receptacle, the reservoir move from the first to the second position;

FIGS. 6A and 6B very schematically illustrate still another alternative embodiment of the means for having, when the cartridge is in the receptacle, the reservoir move from the first to the second position;

FIGS. 7A and 7B very schematically illustrate still another alternative embodiment of the means for having, when the cartridge is in the receptacle, the reservoir move from the first to the second position.

### DETAILED DESCRIPTION

The same elements have been designated with the same reference numerals in the different drawings. For clarity, only those elements which are useful to the understanding of the present invention have been shown and will be described. In particular, the forming of the application part of felt, membrane, or the like type has not been detailed, the described embodiments being compatible with usual application or diffusion parts. Further, the invention will be described hereafter in relation with a hand-held perfume application device. The device and the recharge for which embodiments will be described however more generally apply to any volatile or pasty liquid capable of being applied or diffused by means of a part of felt or wick type.

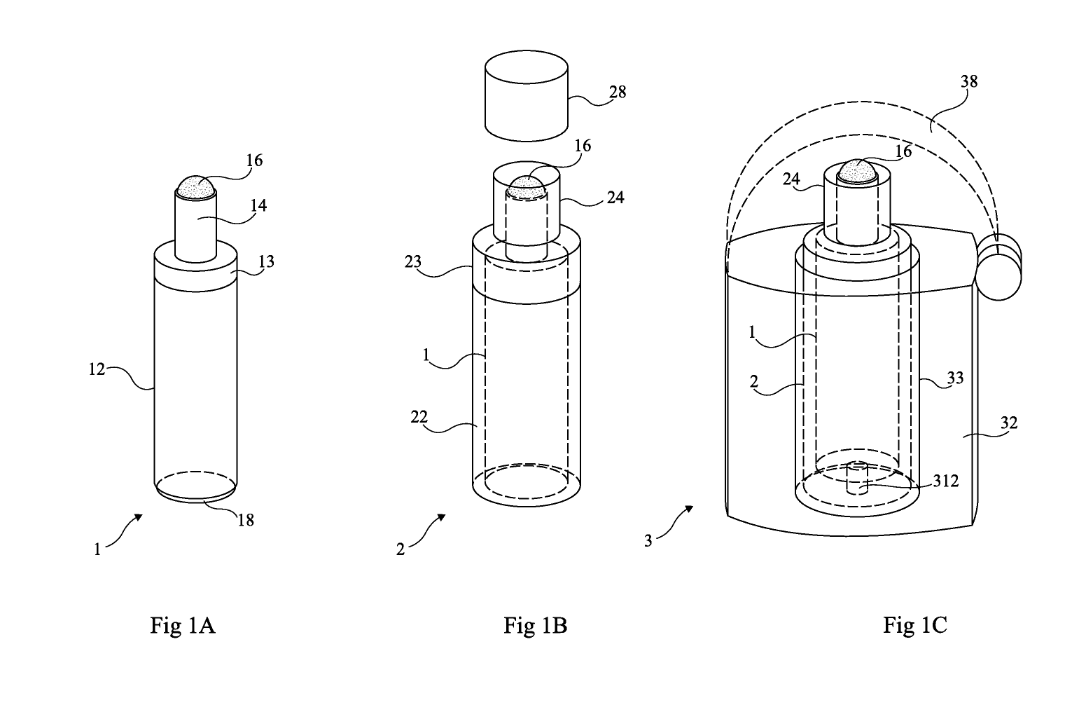

FIGS. 1A, 1B, and 1C are simplified perspective views of an embodiment of components of a perfume applicator device.

This device comprises three sub-assemblies: a product reservoir 1 associated with an application or diffusion part, a cartridge 2 for receiving reservoir 1 and a receptacle 3 or case of cartridge 2 enabling to use the product

FIG. 1A shows reservoir 1 formed of an approximately cylindrical body 12 with a flat bottom having the perfume (liquid or pasty) packaged therein. Body 12 is closed in its upper portion by a plug 13 defining a neck 14 for receiving a felt part 16. Felt 16 protrudes from the free end of neck 14 to enable, as will be seen hereafter, to apply the product Reservoir 1 preferably is a disposable reservoir irreversibly closed on manufacturing.

In the example shown in the drawings, the bottom of reservoir 1 supports a metal pellet or disk 18 covering all or part of the bottom. The pellet is for example glued to the bottom of the plastic reservoir, overmolded therein, inserted in a cut area, etc.

FIG. 1B is a view showing, in dotted lines, reservoir 1 housed in cartridge 2.

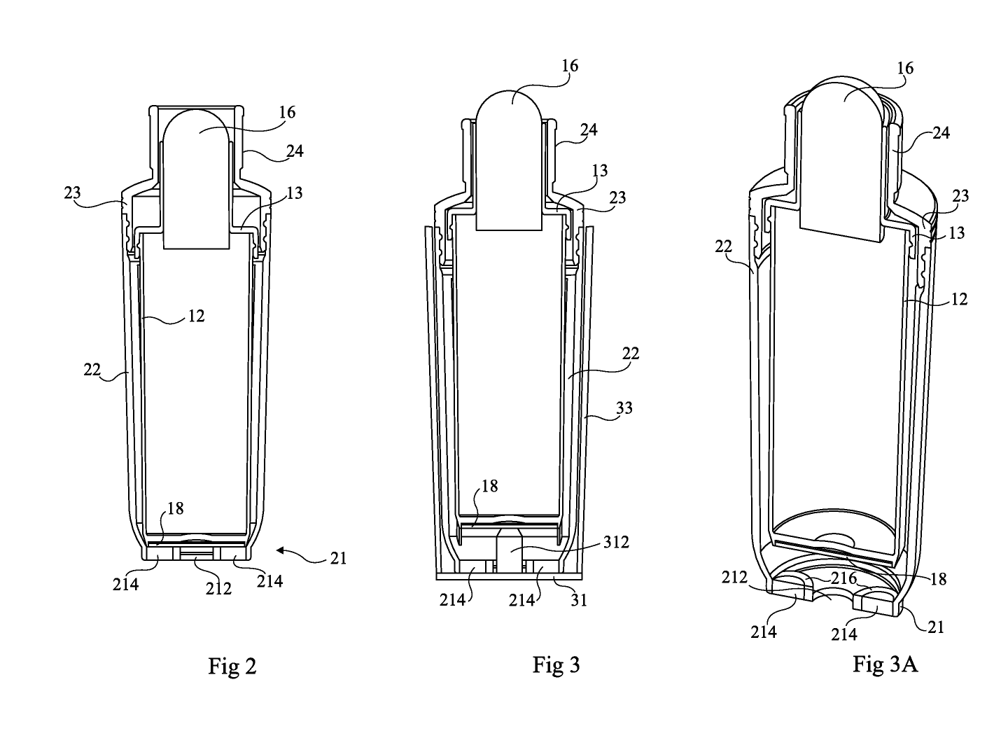

Cartridge 2 comprises a body 22 having, for example, a cylindrical general shape, capable of receiving reservoir 1. In its upper portion (in the orientation of the drawings), body 22 is capable of being closed by a plug 23 ending in a neck 24. The shape and the dimensions of neck 24 are adapted to receiving neck 14 of the reservoir. The dimensions of cartridge 2 are such that, when reservoir 1 rests on the bottom of cartridge 2 (FIG. 2), felt 16 does not protrude from neck 24 and is thus not accessible. Instead of directly resting on the bottom of the cartridge, it is possible for the reservoir to rest thereon via spacers, pins, or the like.

FIG. 1C is a view illustrating cartridge 2 provided with reservoir 1 in receptacle 3.

Receptacle 3 defines an external shape selected according to the application to enable to user to handle the device. Body 32 of receptacle 3 comprises a shaft 33 for receiving cartridge 2 and more particularly its body 22.

Receptacle 3 comprises means 312 so that, when cartridge 2 is engaged all the way to the bottom of the shaft, reservoir 1 contained by the cartridge is driven upwards (in the orientation of the drawings) to make felt 16 accessible outside of neck 24.

Thus, the dimensions (and especially the height) of cartridge 2 are selected so that reservoir 1 can move therein between a retracted position where felt 16 does not come out of neck 24 and an extended position where felt 16 is accessible, the free end of neck 14 being preferably flush with the end of neck 24.

In the embodiment illustrated in FIG. 1C, receptacle 3 comprises a cover 38 capable of protecting felt 16 when it is not being used.

Similarly, cartridge 2, before being introduced into receptacle 3, is preferably associated with a cap 28 engaging on neck 24 to avoid for the upper portion of felt 16 to be exposed to the open air.

FIGS. 2, 3, and 3A illustrate an embodiment of means for vertically positioning reservoir 1 in cartridge 2, FIG. 3A being a perspective and cross-section view of FIG. 3.

In this embodiment, bottom 21 of cartridge 2 comprises a through opening 212, preferably centered, and at least one magnet 214 capable of cooperating with disk 18 of reservoir 1. Two magnets 214 diametrically opposite with respect to opening 212 are for example provided for reasons of symmetry. As a variation, a ring-shaped magnet or a different number of point magnets may be provided around opening 212.

In the shown embodiments, it is assumed that the reservoir, the cartridge, and the receptacle are essentially made of plastic. It is then provided to include magnets 214 in housings 216 (FIG. 3A) formed in bottom 21. As a variation, reservoir 1 is made of magnetizable metal and cooperates with magnets 214.

In a simplified embodiment, housings 216 only emerge on the internal side of the cartridge to cooperate with disk 18.

In the preferred embodiment, as shown, housings 216 emerge on either side of bottom 21 to cooperate, as will be seen hereafter, with receptacle 3.

When it is in cartridge 2 and the latter is not in receptacle 3, reservoir 1 is attracted towards the bottom of cartridge 2 by the magnetization of disk 18 on magnets 214. Felt 16 is then protected from the outside and does not emerge from neck 24.

Bottom 31 of receptacle 3 comprises a pin or finger 312 intended to cooperate with opening 212 of cartridge 2. More specifically, pin 312 is intended to cross opening 212 to bear against the bottom of reservoir 1 and to push it towards the top of cartridge 2. As illustrated in FIGS. 1C, 3, and 3A, cartridge 1 then is in a position where felt 16 has come out of neck 24 and can be used.

Preferably, the height of pin 312 is adapted so that head or plug 13 of the reservoir bears against the inside of head or plug 23 of cartridge 2 and the reservoir no longer moves. Preferably, reservoir 1 in this position cannot rotate in cartridge 2, which eases the application of the perfume. Preferably, the same occurs between cartridge 2 and receptacle 3 having its shaft 33 comprising, for example, ribs preventing a rotation of the cartridge.

Preferably, bottom 31 of receptacle 3 (and possibly pin 312) is totally or partially metallic, to cooperate with magnets 214. Thus, when cartridge 2 is introduced into shaft 33 of the receptacle, it is attracted towards the bottom by magnets 214. The cartridge is then removably maintained in the receptacle.

When cartridge 2 is automatically pushed out of receptacle 3 by the magnetization between parts 18 and 214, reservoir 1 moves down to the bottom of cartridge 2.

The use of a pin 312 cooperating with an opening 212 to push back up the reservoir into the cartridge is a particularly simple and preferred embodiment. Other equivalent means may however be provided.

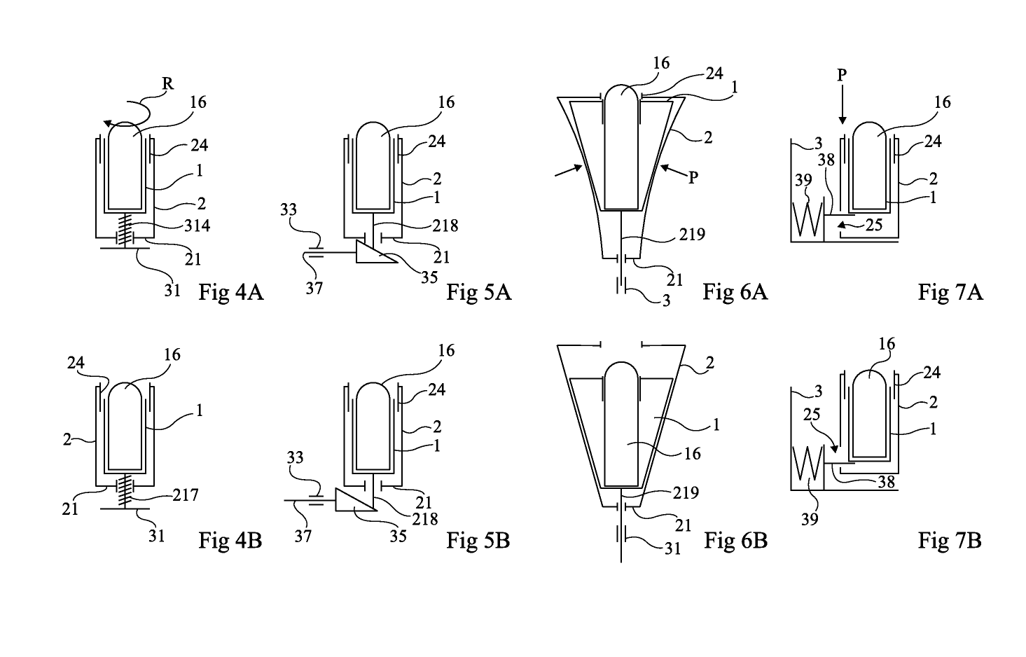

FIGS. 4A to 7B are simplified representations of alternative embodiments of the means for pushing out diffusion part 16 via an action on the reservoir.

In all these variations, the diffuser is arbitrarily shown as occupying the entire height of reservoir 1. This may also correspond to a variation. Figure A shows the system in the second position (out) and Figure B shows the system in the first position (retracted).

FIGS. 4A and 4B illustrate a variation according to which pin 314 crossing the bottom of the receptacle is threaded and cooperates with a tapping of opening 212 of the cartridge. Screwing R brings bottom 21 closer to bottom 31, which pushes the reservoir upwards and pushes diffuser 16 out of neck 24 of the cartridge.

FIGS. 5A and 5B functionally illustrate another variation according to which the upward pushing of reservoir 1 is performed by means of a wedge 35 or the like having its inclined surface cooperating with a vertical rod 218 connected to the bottom of the reservoir and crossing bottom 21 of the cartridge. Thus, a horizontal translation motion of wedge 35 pushes diffuser 16 out. Wedge 35 is for example housed in a false bottom of receptacle 3, a member (for example, a rod 37) crossing wall 33 of the receptacle to be accessible from the outside and to be able to control its horizontal sliding.

FIGS. 6A and 6B illustrate still another variation according to which reservoir 1 has a conical shape (pointing downwards) and the cartridge has a matching shape, but in a material more flexible than the reservoir. Thus, a pressure P on the sides of the cartridge causes an upward motion of the reservoir, and thus of the diffuser. A rod crossing the bottom of the cartridge and, for example, bottom 31 of the receptacle, provides an axial guiding. The action on the reservoir walls is for example performed through ports (not shown) formed in the receptacle.

FIGS. 7A and 7B illustrate still another embodiment according to which, as the cartridge engages into receptacle 3, a horizontal finger 38 pressed by a spring 39 against the vertical wall of receptacle 3 may penetrate into a vertical port 25 of the cartridge and come under the bottom of reservoir 1. The pressure of spring 39 is maintained as long as the cartridge is not in the receptacle. When the cartridge is engaged, the finger penetrates into the port and passes under the bottom of the reservoir (the reservoir and the cartridge are sized so that the reservoir does not rest on the bottom of the cartridge with its entire surface and allows the passing of finger 38. Then, a vertical pressure P applied on the cartridge causes the extraction of the diffuser.

According to another embodiment, not shown, the means for pushing out diffusion part 16 via an action on reservoir 1 are accessible from the outside of the receptacle. It may be a rod, a ring, a disk, etc. controllable at will from the outside, for example, by means of a knob, or a cursor parallel to the reservoir, or the like. Providing means capable of being actuated by a control member accessible from the outside of the receptacle adds a functionality available for the user.

Preferably, reservoir 1, cartridge 2, and shaft 33 are of generally cylindrical shape with a circular transverse cross-section, which eases both their manufacturing and their use. Preferably, cartridge 2 and shaft 33 are, in longitudinal cross-section, slightly conical to ease the introduction and the pushing out of cartridge 2. However, any other shape can be envisaged. For example, cartridges having oval, or even square or polygonal cross-sections may be provided, provided to adapt the shaft of receptacle 3. Similarly, the internal wall of cartridge 2 is adapted to the external wall of reservoir 1 and may have an internal cross-section with a different shape than its external cross-section.

In the example of FIGS. 2 and 3, a felt-shape application element 16 having its lower end in contact with the inside of reservoir 1 is considered. Other embodiments are possible, for example, a membrane associated with a wick dipped into the reservoir or any other system for diffusing or applying similar liquid or pasty products.

The device discussed hereabove enables to refill receptacle 3 with removable, disposable, or interchangeable cartridges 2. Preferably, cartridges 2 are single-use rather than rechargeable.

In an example of application to a perfume applicator device, a same receptacle 3 for example forming a pocket or purse applicator, is capable of receiving different cartridges 2.

Further, the number of fingers 312 arranged at the bottom of the receptacle to push the reservoir upwards may be greater than 1, provided to be adapted to the number of openings of the bottom of the cartridge.

In the above embodiment, the product contained in the cartridge is not usable when it is not housed in the receptacle, the felt being inaccessible to be used as an applicator.

Various embodiments have been described, various alterations and modifications are within the abilities of those skilled in the art. In particular, the materials forming the receptacle, the cartridge, and the reservoir may be modified provided to respect the described functionalities. For example, the receptacle may be coated with wood or metal or be totally or partly made of these materials to provide a fine appearance. Similarly, the cartridge and the reservoir may be made of another material than plastic, provided, in the preferred embodiment, to be compatible with the described operation by magnetization. Further, it may be provided for disk 18 and pin 312 to be magnetized and for magnets 212 to be replaced with metal elements.

1. An applicator device for liquid or volatile products comprising:
a product reservoir having a neck containing a part for applying or diffusing the product;
a cartridge for receiving the reservoir, provided with a neck, the cartridge being adapted for the reservoir to take a first position where the application or diffusion part does not protrude from the neck, and a second position where this part protrudes from the neck; and
a receptacle for the cartridge, provided with means for having, when the cartridge is in the receptacle, the reservoir move from the first to the second position.

2. The device of claim 1, wherein said means comprise at least one pin protruding from a shaft comprised in the receptacle, the pin being intended to cross an opening of the cartridge to drive the reservoir to the second position.

3. The device of claim 2, wherein the bottoms of the cartridge and of the shaft of the receptacle comprise elements capable of cooperating by magnetization when the reservoir is in the second position.

4. The device of claim 2, wherein said pin is threaded, said opening of the cartridge being tapped.

5. The device of claim 1, wherein said means comprise a horizontally-mobile wedge capable of cooperating with a rod protruding from the bottom of the reservoir and crossing the bottom of the cartridge.

6. The device of claim 1, wherein the reservoir and the cartridge have a conical shape, the cartridge wall being more flexible than the wall of the reservoir so that a pressure on the cartridge wall causes an axial displacement of the reservoir.

7. The device of claim 1, wherein said means comprise a horizontally-mobile pin capable of crossing a vertical port of the cartridge to bear against the bottom of the reservoir.

8. The device of claim 1, wherein said means can be actuated by a control member accessible from the outside of the receptacle.

9. The device of claim 1, wherein the bottoms of the reservoir and of the cartridge comprise elements capable of cooperating by magnetization when the reservoir is in the first position.

10. The device of claim 9, wherein the bottom of the cartridge comprises at least one magnet, the bottom of the reservoir comprising at least one metal part opposite to this magnet

11. The device of claim 10 wherein the bottoms of the cartridge and of the shaft of the receptacle comprise elements capable of cooperating by magnetization when the reservoir is in the second position, and said magnet is housed in a through opening of the bottom of the cartridge and is capable of cooperating with a metal bottom of the shaft of the receptacle.

12. The device of claim 1, wherein the reservoir, the cartridge, and the shaft of the receptacle have an approximately cylindrical shape.

13. The device of claim 1, wherein application or diffusion part is a felt.

14. The device of claim 1, wherein the reservoir contains perfume.

15. The device of claim 1, wherein the receptacle comprises a removable cover.

16. The device of claim 2 wherein the bottoms of the reservoir and of the cartridge comprise elements capable of cooperating by magnetization when the reservoir is in the first position.

17. The device of claim 3 wherein the bottoms of the reservoir and of the cartridge comprise elements capable of cooperating by magnetization when the reservoir is in the first position

18. The device of claim 6, wherein said means comprise a horizontally-mobile wedge capable of cooperating with a rod protruding from the bottom of the reservoir and crossing the bottom of the cartridge.

19. The device of claim 8, wherein said means comprise a horizontally-mobile wedge capable of cooperating with a rod protruding from the bottom of the reservoir and crossing the bottom of the cartridge.

20. The device of claim 12, wherein said means comprise a horizontally-mobile wedge capable of cooperating with a rod protruding from the bottom of the reservoir and crossing the bottom of the cartridge.
